(19) United States
(12) Patent Application Publication
(10) Pub. No.: US 2017/0340491 A1
(43) Pub. Date: Nov. 30, 2017

(54) Apparatus and method to protect an injury and/or a surgical wound

(71) Applicant: Plymouth State University, Plymouth, NH (US)

(72) Inventors: Julie Bernier, Plymouth, NH (US); Joan Andresen, Plymouth, NH (US)

(21) Appl. No.: 15/609,503

(22) Filed: May 31, 2017

Related U.S. Application Data
(60) Provisional application No. 62/392,387, filed on May 31, 2016.

(51) Int. Cl. A61F 15/00; A61F 13/00; A61F 13/14

(52) U.S. Cl. CPC A61F 15/004; A61F 13/148; A61F 13/00017; A61F 13/00029

(57) ABSTRACT

A protector assembly for protecting an injury and/or surgical wound includes a protector shell having a shell front side, a shell back side and shell circumferential edge where the protector shell has a size and shape that is larger than the injury and/or surgical wound and shaped to correspond to a portion of the body having the injury and/or surgical wound to thereby protect the injury and/or surgical wound from further injury. The protector assembly also includes a protector pad having a pad front side, a pad back side and a pad circumferential edge wherein the protector pad has a pad opening sized to circumscribe the injury and/or surgical wound, the pad front side being attached to the shell back side of the protector shell where the pad back side is between the injury and/or surgical wound and the protector shell.

### BACKGROUND OF THE INVENTION

### 1. Field of the Invention

The present invention relates generally to an apparatus and method to protect areas of the human body. Particularly, the present invention relates to an apparatus and method to protect areas of the body that have been injured or subject to surgical procedures.

### 2. Description of the Prior Art

Surgery normally results in a wound on the outside of the body. An individual who returns home after an injury or surgical procedure is vulnerable to unintentional further injury. Such unintentional injury is commonly caused by excited and unaware children and pets. For example, many post-surgical patients are sent home with gauze bandages or at most, light padding. The abdomen is particularly vulnerable post-surgically to unintentional injury. Dogs jump on their owners; children run and jump into the lap of family members; cats climb onto their owner's abdomen or chest while they sleep; and vehicle seatbelts because the belt often goes right across the surgical incision.

There have been devised several devices that are designed to protect an injury and/or wound on an individual. The following are examples of such devices.

U.S. Pat. No. 5,062,433 (1991, Kummer) discloses a protector pad to prevent hip fracture and/or bed sores and/or protect a wound or wound area. The protector pad is formed from an outer load bearing member shaped with structured ridges and is dome shaped with structured ridges to form an internal dome or clearance for the region of desired protection. A soft inner sheet is attached for comfort. The outer shell formation distributes the load or pressure to the edges of the device, protecting the organ covered by the dome.

U.S. Pat. No. 4,399,816 (1983, Spangler) discloses a wound protector with transparent cover. The wound protector includes a base pad of a pliable, fibrous or foamed non-allergenic material in an angular form and a central opening that is larger than the wounded area about which it is to be placed. A marginal edge is formed in the top surface of the base pad around the inner opening. The inner opening may optionally receive a surgical gauze or sponge containing a medicament. A top surface of the base pad between the marginal ledge and the outer vertical surface of the pad must be of sufficient width to support a transparent cover. The transparent cover is of the same shape as the base pad and is secured at one side by a permanent adhesive material.

U.S. Pat. No. 2,367,690 (1942, Purdy) discloses a wound protector. The wound protector includes a flexible base having a central aperture and an adhesive coated under surface, a relatively rigid frame of lesser area than the base, which frame is bonded to an upper surface of the flexible base where the inner margins of the base and frame are in substantial registry with each other. The wound protector further includes an upstanding flange at the inner margin of the frame, a transparent cover telescopically fitted over the flange of the frame, means for releasably retaining the cover in closed relation to the frame, and auxiliary means for retaining the protector in place. The auxiliary means consists of band sections anchored to opposite edge portions of the flexible base.

### SUMMARY OF THE INVENTION

Many individuals who have injured areas on their bodies are subject to further unintentional injury through the inadvertent actions of children and/or pets or from a seatbelt in motor vehicle while riding in the vehicle. This unintentional injury may delay healing of the original injury and/or surgical wound. For example, in the case of a surgical wound, stitches holding the incision or cut closed may be re-opened by the unintentional injury causing more pain and delayed recovery.

It is an object of the present invention to provide an apparatus and method to prevent an injury and/or surgical wound from incurring further unintentional injury.

The present invention achieves these and other objectives by providing a thermally moldable hard shell to protect the injured area from further injury. It has an interior lining consisting of foam padding. An area of the foam padding slightly larger than the injured area is removed. The shell is then placed over the foam padding and over the injured area on an individual and held in place against the injury and/or surgical wound and secured in place.

In one embodiment, the present invention is a protector assembly having a protector shell and a protector pad where the protector assembly is a protector assembly having a protector shell and a protector pad where the protector assembly configured for the lower abdomen.

In another embodiment, the present invention is a protector assembly having a protector shell and a protector pad where the protector assembly is a protector assembly having a protector shell and a protector pad where the protector assembly configured for the upper abdomen or chest area.

In one embodiment of the present invention, a protector assembly for protecting an injury and/or surgical wound includes a protector shell having a shell front side, a shell back side and shell circumferential edge where the protector shell has a size and shape that is larger than the injury and/or surgical wound and shaped to correspond to a portion of the body having the injury and/or surgical wound to thereby protect the injury and/or surgical wound from further injury. The protector assembly also includes a protector pad having a pad front side, a pad back side and a pad circumferential edge where the protector pad has a pad opening sized to circumscribe the injury and/or surgical wound. The pad front side is attached to the shell back side of the protector shell. The pad back side is between the injury and/or surgical wound and the protector shell.

In one embodiment of the present invention, the protector assembly has a protector pad that is removable from the protector shell.

In another embodiment of the present invention, the protector shell is made of a thermoplastic material.

In still another embodiment, the protector shell is made of acrylonitrile-butadiene-styrene, an acrylic-polyvinyl chloride material, or a splint making material.

In yet another embodiment, the protector shell has a thickness of about 0.060 to 0.125 inches.

In a further embodiment, the protector pad is foam pad.

In one embodiment, the protector pad is a high-density, closed-cell foam pad.

In one embodiment, the protector pad has a thickness in the range of about 0.5 inches to about 0.75 inches.

In one embodiment, the protector assembly further includes at least one of (1) a strap that surrounds the protector assembly and maintains the protector assembly in place over the injury and/or surgical wound, and (2) an abdominal binder between the injury and/or surgical wound and the protector pad. In another embodiment, the abdominal binder is a Dale abdominal binder.

In another embodiment, there is disclosed a method of protecting an injury and/or surgical wound of an individual from further injury. The method includes providing a protector assembly having a protector shell and a protector pad attached to a back side of the protector shell. The protector assembly has a size and shape commensurate with a size and location of the injury and/or surgical wound. The method also includes removing a portion of the protector pad from a pad back surface that will coincide with an area of the injury and/or surgical wound, placing the protector assembly against the injury and/or surgical wound so that the removed portion of the protector pad coincides with the injury and/or surgical wound, and securing the protector assembly to the individual.

In another embodiment, the method includes providing a protector assembly having a protector pad that is removable from the protector shell.

In one embodiment, the method includes providing a protector assembly to the individual that includes attaching an abdominal belt to the individual and attaching a back side of the protector pad to the abdominal belt.

### BRIEF DESCRIPTION OF THE DRAWINGS

FIG. 1 is a front elevation view of one embodiment of the present invention showing the protector assembly attached to a person's lower abdomen.

FIG. 2 is a front elevation view of another embodiment of the present invention showing the protector assembly attached to a person's upper abdomen and/or chest.

FIG. 3 is a perspective view of one embodiment of the protector assembly of the present invention.

FIG. 4 is an expanded view of one embodiment of the protector assembly of the present invention showing the protector shell and the protector pad.

FIG. 5 is a front view of one embodiment of the protector shell of the present invention.

FIG. 6 is a right side view of the protector shell of FIG. 5.

FIG. 7 is a left side view of the protector shell of FIG. 5.

FIG. 8 is a back side view of the protector shell of FIG. 5.

FIG. 9 is a front view of one embodiment of the protector pad of the present invention.

FIG. 10 is a right side view of the protector pad of FIG. 9.

FIG. 11 is a left side view of the protector pad of FIG. 9.

FIG. 12 is a back side view of the protector pad of FIG. 9.

### DETAILED DESCRIPTION OF THE INVENTION

The present invention claims the benefit of U.S. Provisional Patent Application No. 62/392,387, filed May 31, 2016, which is incorporated herein by reference.

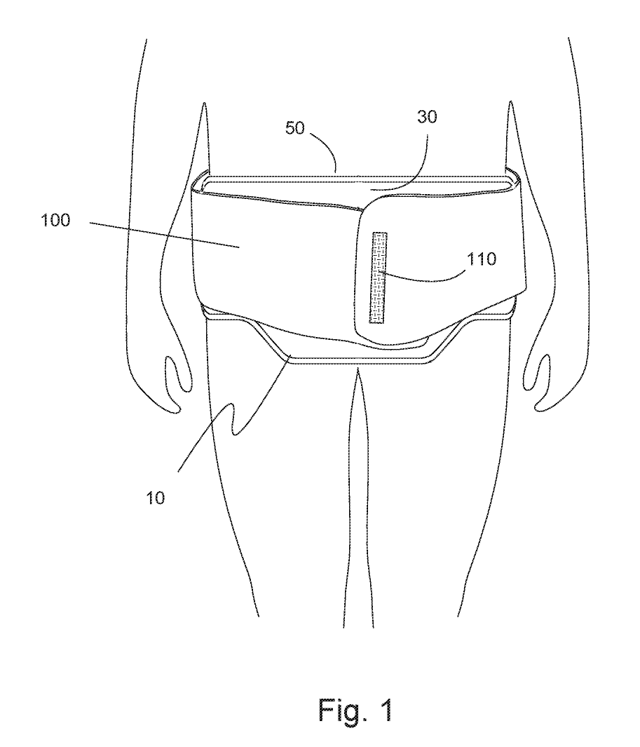

Embodiments of the present invention are illustrated in FIGS. 1-12. FIG. 1 shows one embodiment of a protector assembly 10. Protector assembly 10 includes a protector shell 30 and a protector pad 50 attached to protector shell 30. Protector assembly 10, in this embodiment, is disposed on a lower abdomen of an individual and retained in place by a removable strap 100. Removable strap 100 is wrapped around the individual's abdomen and secured to itself by a connector 110. Removable strap 100 may be a neoprene belt with a hook and loop closure or may be made of any type of 6 inch (15.2 cm) to 8 inch (20.3 cm) wide stretch fabric with hook and loop closures. Connector 110 may be any of the type typically used in the medical field such as clips, hook and loop fasteners, and the like that does not require excessive force since the protector assembly 10 is used to protect an injury or surgical wound of the individual and should not cause further pain when attaching the protector assembly to the individual.

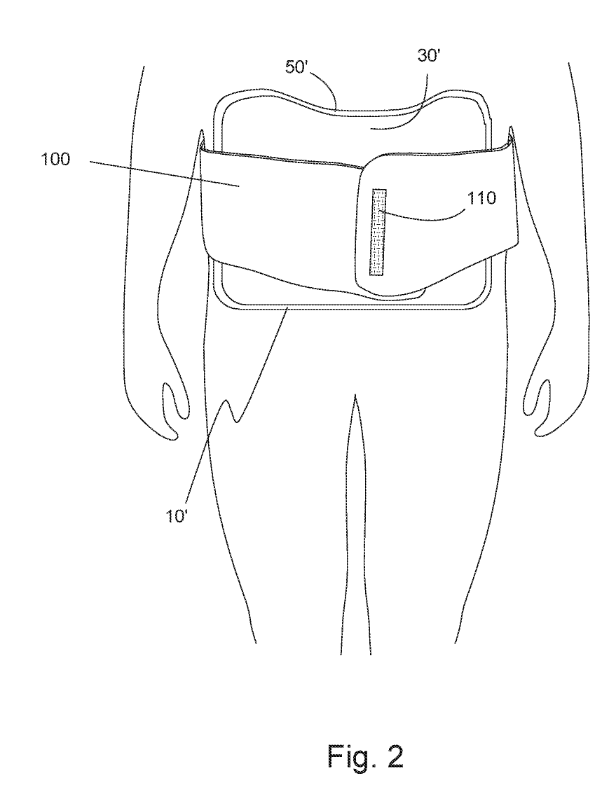

FIG. 2 illustrates another embodiment of the protector assembly 10′. Like protector assembly 10, protector assembly 10′ includes a protector shell 30′ and a protector pad 50′ attached to protector shell 30′. Protector assembly 10′, in this embodiment, is disposed on an upper abdomen of an individual and retained in place by removable strap 100. Removable strap 100 is wrapped around the individual's chest and secured to itself by connector 100. Protector assembly 10′ is used to protect the chest such as, for example, after mastectomy surgery. It is contemplated that protector assembly 10, 10′ may be worn on top of or under clothing. It is also contemplated that protector assembly 10, 10′ may be made in multiple sizes and shapes to accommodate use on adults, children and other animals such as in veterinary medicine as well as to protect other areas of the body and not just the abdomen.

Turning now to FIG. 3, the protector assembly 10 is shown. In one embodiment, protector pad 50 is attached to protector shell 30 by adhesives, hook and loop fasteners and the like. As can be seen in FIG. 3, the protector assembly 10 has a curved shape configured to fit over an abdomen where protector pad 50 maintains a predefined spacing between protector shell 30 and the abdomen of the individual. Protector shell 30 is a hard shell made of a firm, thermally moldable plastic. In one embodiment, protector shell 30 is one-eighth inches (⅛″) (about 3.2 mm) thick acrylonitrile-butadiene-styrene (ABS) or a thermoplastic acrylic-polyvinyl chloride material sold under the trademark KYDEX® and manufactured by Sekisui SPI, Bloomsburg, Pa. Material used for splints such as that sold under the trademark Orthoplast® may also be used to form protector shell 30.

In one embodiment of protector pad 50, protector pad 50 is a layer of foam padding. The foam padding is a high-density, closed-cell foam material. Examples of such foam padding can be found in yoga mats or sleeping pads. Protector pad 50 has a preferred thickness the range of 0.5 inches (12.7 mm) to 0.75 inches (19 mm).

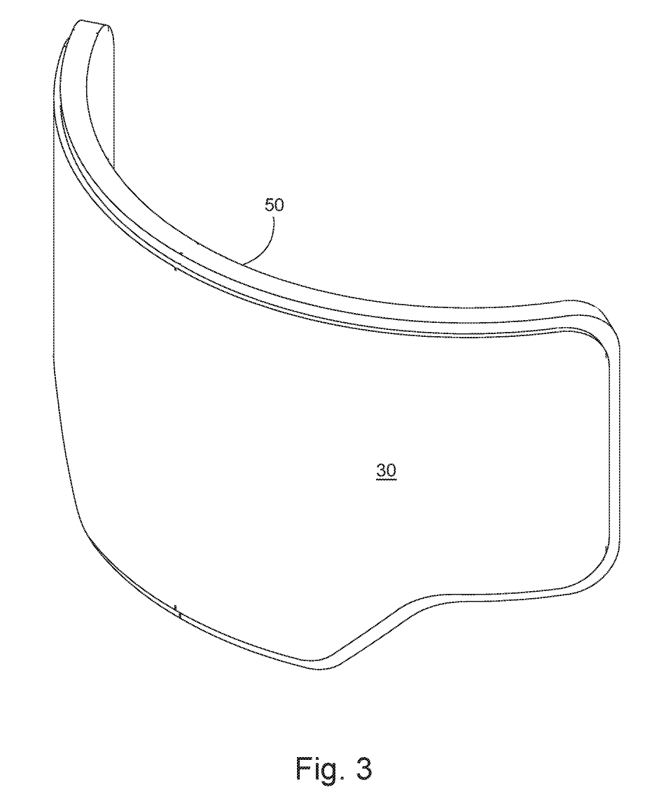

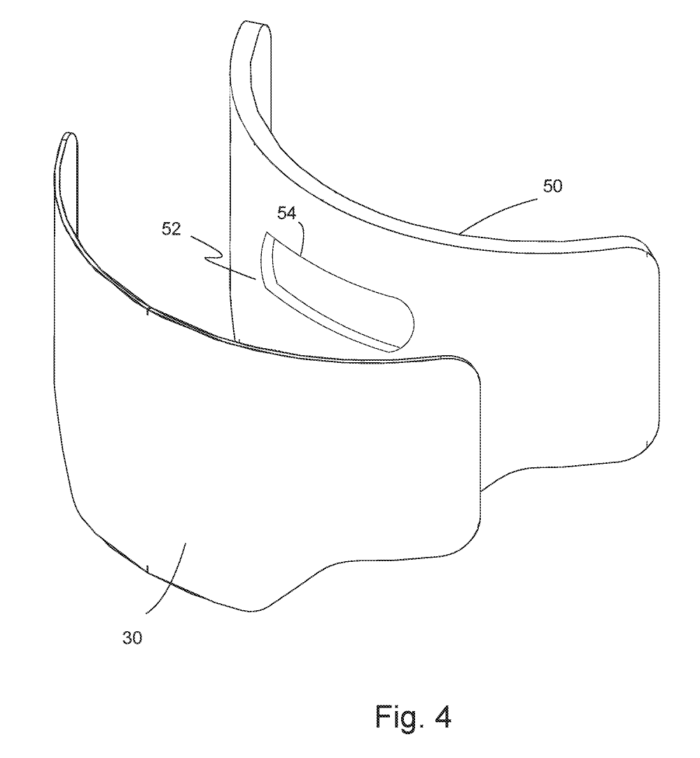

FIG. 4 illustrates an exploded view of protector assembly 10. In this view, a portion 52 of protector pad 30 is cutout/removed creating a pad opening 54. The shape and size of pad opening 54 depends on the size and shape of the injury or surgical wound of the individual. Typically, pad opening 54 has an area that is approximately 0.5 inches larger around the injury and/or surgical wound or incision. For example, if the individual has a surgical wound (i.e. stitches in the abdomen from surgery), then opening 54 may have an elongated shape so that the stitches are surrounded by opening 54 with about 0.5 inches between the wound/incision or injury area and an edge of opening 54. The shape of opening 54 may be any shape that prevents protector pad from contacting the injury or surgical wound such as oblong, elliptical, square, rectangular, circular, trapezoidal, T-shaped, C-shaped, D-shaped, or any other shape required to provide pain-free protection of the injured or surgical wound area.

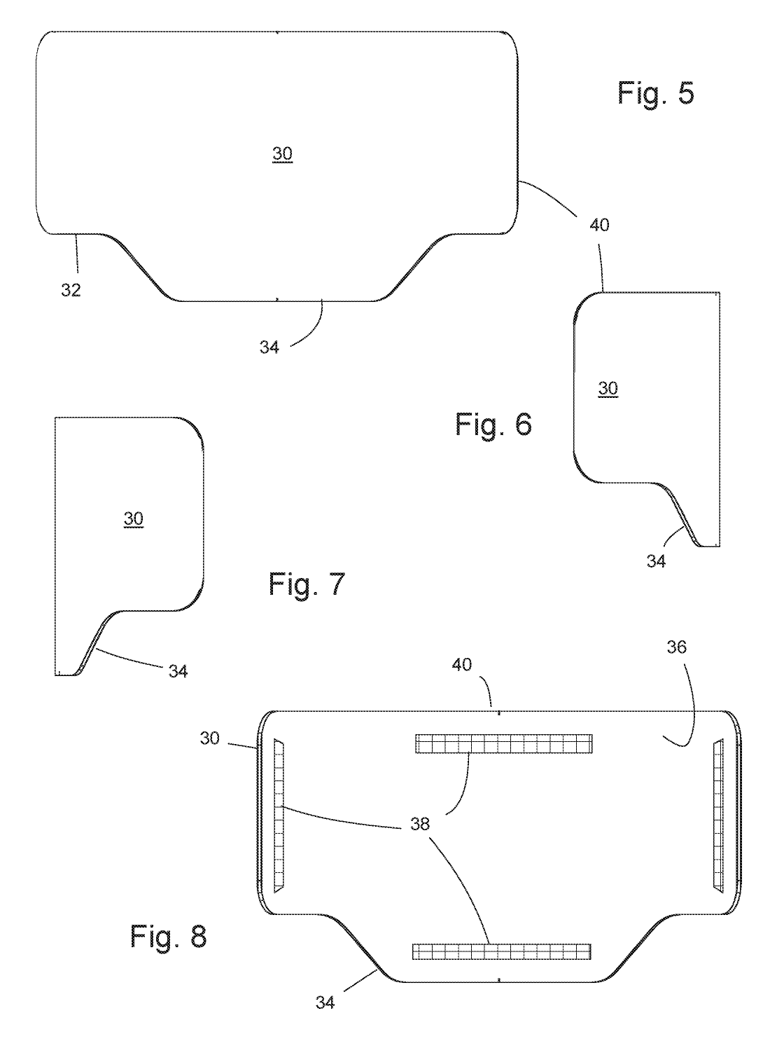

FIG. 5 is a front view of protector shell 30. As can be seen, protector shell 30 is shaped for positioning over an individual's lower abdomen. Protector shell 30 in this embodiment has a generally curved, rectangular shape with an extension portion 34 along a long side 32 for covering the abdomen adjacent the legs of the individual. FIG. 6 is a right side view of protector shell 30 shown in FIG. 5. The contoured feature and the curved shape of protector shell 30 provide a comfort fit in the area of the lower abdomen. FIG. 7 is a left side view of protector shell 30. FIG. 8 is a back view of protector shell 30 shown in FIG. 5. In this embodiment, a shell back surface 36 has an optional feature that is a plurality of strips 38 of one portion of a hook and loop fastener to allow protector pad 50 to be removable from protector shell 30.

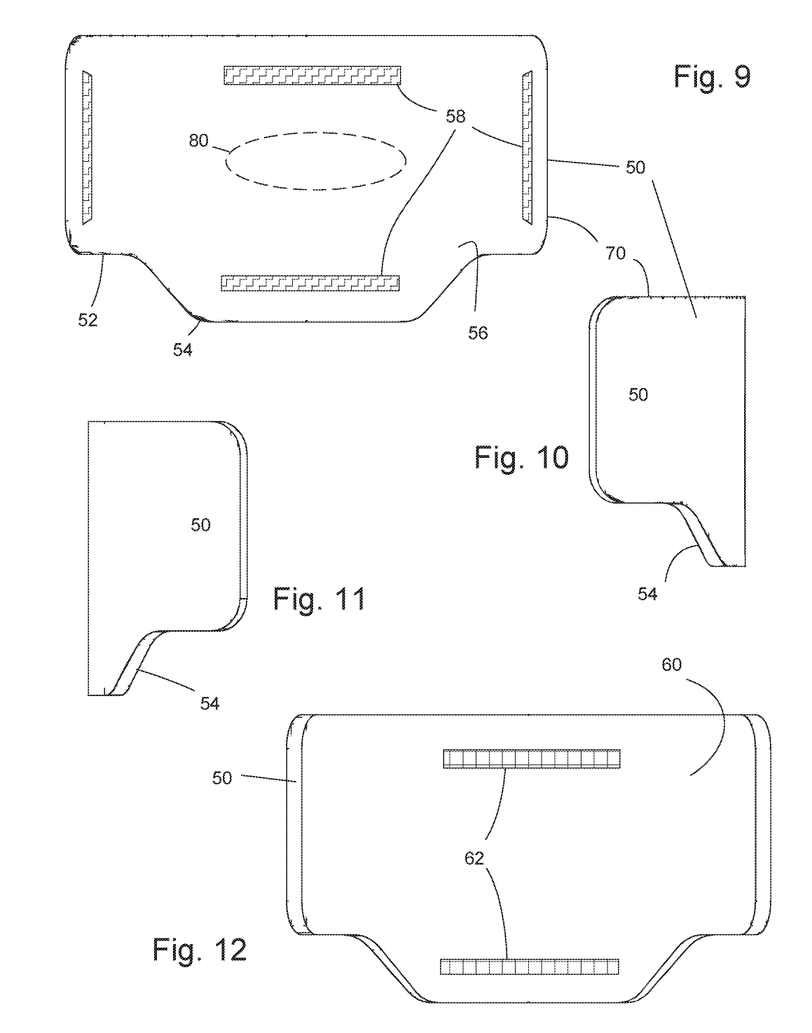

Turning now to FIGS. 9 to 12, there is illustrated various views of one embodiment of protector pad 50. FIG. 9 is a front view of protector pad 50. Protector pad 50 in this embodiment has a generally curved, rectangular shape with an extension portion 54 along a long side 52 for covering the abdomen adjacent the legs of the individual. The shape of protector pad 50 coincides with the shape of protector shell 30, however, protector pad 50 is larger than protector shell 30 so that a pad circumferential edge 70 of protector pad 50 extends beyond a shell circumferential edge 40 so that no part of shell circumferential edge 40 touches the abdomen of the individual. Also shown in FIG. 9 is an outline 80 of an elliptical shape that is cutout of protector pad 50 to create pad opening 54. In this embodiment, a pad front surface 56 has a feature that includes a plurality of pad strips 58 positioned to coincide with the plurality of shell strips 38 on shell back surface 36 when protector pad 50 is configured for removable instead of permanent attachment to protector shell 30. In this embodiment, pad front surface 56 with the plurality of pad strips 58 allow protector pad 50 to be removable from protector shell 30. The skilled artisan understands that the plurality of pad strips 58 (FIG. 9) are the mating portion of the hook and loop fastener to the portions of the hook and loop fastener on the back surface 36 of protector shell 30 (FIG. 8).

FIG. 10 is a right side view of protector pad 50 shown in FIG. 9. The contoured feature and the curved shape of protector pad 50 provide a comfort fit against the lower abdomen. FIG. 11 is a left side view of protector pad 50. FIG. 12 is a back view of protector pad 50 shown in FIG. 9. In this embodiment, a back pad surface 60 has a plurality of back pad strips 62 of the hook portion of a hook and loop fastener for attachment to an abdominal binder (not shown) such as a Dale binder when a Dale binder is used instead of strap 100.

The method of making protector assembly 10 of the present invention will now be discussed. In one embodiment, the protector shell 30 and the protector pad 50 is preformed without the need to heat and mold the protector shell 30. In such an embodiment, the protector assembly 10 may include multiple sizes to accommodate various body sizes and types as well as injuries and/or surgical wounds in parts of the body other than the abdomen. The protector pad 50 is removably attached to protector shell 50 using a fastener system such as, for example, hook and loop fasteners. In another embodiment, the protector shell 30 may be heated to create a custom fit. In this case, the foam padding is removed and the thermoplastic protector shell 30 is heated in an oven or in hot water depending on the type of material used for protector shell 30 and preformed to the desired curvature. To accomplish this, the heated protector shell 30 is placed over the protective foam pad 50 forming the protector assembly 10, which is loosely strapped to the body of the individual while the heated protector shell 30 cools and forms to the portion of the body to which it is loosely strapped.

The individual or a clinician removes a predefined area of protector pad 50 forming pad opening 54 that is larger than the injured area or the surgical wound/incision. The individual or clinician may optionally separate protector pad 50 from protector shell 30 to remove the predefined area of protector pad 50 creating pad opening 54. Typically, the area is removed by cutting the protector pad with a sharp object such as a razor blade, surgical knife, Exacta® tool, and the like.

In another embodiment, the protector pad 50 may include a plurality of slits through protector pad 50 with frangible connections (not shown) between slits to allow customized removal of portions of protector pad 50 to form pad opening 54.

In either case (i.e. preformed or customized fit), the protector pad 50 in one embodiment is placed over the injured area, followed by the protector shell 30 being secured to protector pad 50. In another embodiment, the protector pad 50 is attached to protector shell 30 forming the protector assembly 10 and protector assembly 10 is placed over the injured area whereby the pad opening 54 coincides with the area of the injury and/or surgical wound. It should be understood that removing a portion of protector pad 50 to form pad opening 54 should be done only after molding protector shell 30.

Although the preferred embodiments of the present invention have been described herein, the above description is merely illustrative. Further modification of the invention herein disclosed will occur to those skilled in the respective arts and all such modifications are deemed to be within the scope of the invention as defined by the appended claims.

What is claimed is:

1. A protector assembly for protecting an injury and/or surgical wound, the protector assembly comprising:
a protector shell having a shell front side, a shell back side and shell circumferential edge wherein the protector shell has a size and shape that is larger than the injury and/or surgical wound and shaped to correspond to a portion of the body having the injury and/or surgical wound to thereby protect the injury and/or surgical wound from further injury; and
a protector pad having a pad front side, a pad back side and a pad circumferential edge wherein the protector pad has a pad opening sized to circumscribe the injury and/or surgical wound, the pad front side being attached to the shell back side of the protector shell;
wherein the pad back side is between the injury and/or surgical wound and the protector shell.

2. The protector assembly of claim 1 wherein the protector pad is removable from the protector shell.

3. The protector assembly of claim 1 wherein the protector shell is made of a thermoplastic material.

4. The protector assembly of claim 3 wherein the thermoplastic material is acrylonitrile-butadiene-styrene, an acrylic-polyvinyl chloride material, or a splint making material.

5. The protector assembly of claim 3 wherein the thermoplastic material has a thickness of about 0.060 to 0.125 inches.

6. The protector assembly of claim 1 wherein the protector pad is foam pad.

7. The protector assembly of claim 6 wherein the foam pad is a high-density, closed-cell foam pad.

8. The protector assembly of claim 1 wherein the protector pad has a thickness in the range of about 0.5 inches to about 0.75 inches.

9. The protector assembly of claim 1 further comprising at least one of (1) a strap that surrounds the protector assembly and maintains the protector assembly in place over the injury and/or surgical wound, and (2) an abdominal binder between the injury and/or surgical wound and the protector pad.

10. A method of protecting an injury and/or a surgical wound of an individual from further injury, the method comprising:
providing a protector assembly having a protector shell and a protector pad attached to a back side of the protector shell, the protector assembly having a size and shape commensurate with a size and location of the injury and/or surgical wound;
removing a portion of the protector pad from a pad back surface that will coincide with an area of the injury and/or surgical wound;
placing the protector assembly against the injury and/or surgical wound so that the removed portion of the protector pad coincides with the injury and/or surgical wound; and
securing the protector assembly to the individual.

11. The method of claim 10 wherein the step of providing a protector assembly includes providing a protector assembly having a protector pad that is removable from the protector shell.

12. The method of claim 10 wherein the step of removing a portion of the protector pad includes removing the protector pad from the protector shell before removing the portion of the protector pad from a pad back surface.

13. The method of claim 10 wherein the step of securing the protector assembly to the individual includes securing with a strap that surrounds the protector assembly and the individual.

14. The method of claim 10 wherein the step of securing the protector assembly to the individual includes attaching an abdominal belt to the individual and attaching a back side of the protector pad to the abdominal belt.
